(19) United States
(12) Patent Application Publication
(10) Pub. No.: US 2024/0268686 A1
(43) Pub. Date: Aug. 15, 2024

(54) METHOD AND APPARATUS FOR SENSING CIRCULATORY HEALTH

(71) Applicants: Steven M. Ebstein, Newton, MA (US); VOTIS Subdermal Imaging Technologies, Ltd., Jerusalem (IL)

(72) Inventors: Roberto Ferraresi, Milano (IT); Steven M. Ebstein, Newton, MA (US)

(21) Appl. No.: 18/642,233

(22) Filed: Apr. 22, 2024

Related U.S. Application Data
(63) Continuation-in-part of application No. 17/711,234, filed on Apr. 1, 2022.
(60) Provisional application No. 63/169,456, filed on Apr. 1, 2021.
(60) Provisional application No. 63/460,707, filed on Apr. 20, 2023.

(51) Int. Cl. A61B 5/021; A61B 5/00; A61B 5/02; A61B 5/022; A61B 5/0295; A61B 5/145; A61B 5/1455; A61B 7/04; A61B 90/00

(52) U.S. Cl. CPC A61B 5/02116; A61B 5/02007; A61B 5/022; A61B 5/0295; A61B 5/14546; A61B 5/14552; A61B 5/7228; A61B 5/7235; A61B 5/7415; A61B 5/742; A61B 90/06; A61B 7/04; A61B 2090/064; A61B 2560/0418; A61B 2560/0443; A61B 2560/0462; A61B 2562/0233; A61B 2562/0247; A61B 2562/0261

(57) ABSTRACT

A probe with a blood circulation sensor and a force or pressure sensor is placed against a patient. One part of the probe applies a force to another part of the probe which is pressed against the patient at one or more locations. The variation of a measure of blood circulation is recorded as a function of the applied pressure, thereby giving the operator a specific knowledge of the Tissue Perfusion Pressure (TPP), a measure of circulatory health, at each location.

(i) is a continuation-in-part of pending prior U.S. patent application Ser. No. 17/711,234, filed Apr. 1, 2022 by VOTIS Subdermal Imaging Technologies, Ltd. and Roberto Ferraresi et al. for PROBE FOR SENSING CIRCULATORY HEALTH (Attorney's Docket No. VOTIS-1), which patent application claims benefit of prior U.S. Provisional Patent Application Ser. No. 63/169,456, filed Apr. 1, 2021 by VOTIS Subdermal Imaging Technologies, Ltd. and Roberto Ferraresi for COMPACT DEVICE FOR SENSING CIRCULATORY HEALTH (Attorney's Docket No. VOTIS-1 PROV); and

(ii) claims benefit of pending prior U.S. Provisional Patent Application Ser. No. 63/460,707, filed Apr. 20, 2023 by VOTIS Subdermal Imaging Technologies, Ltd. and Roberto Ferraresi et al. for PROBE FOR SENSING CIRCULATORY HEALTH (Attorney's Docket No. VOTIS-040506 PROV).

The three (3) above-identified patent applications are hereby incorporated herein by reference.

### FIELD OF THE INVENTION

This invention relates to novel methods and apparatus for atraumatically diagnosing circulatory conditions in subdermal tissue in general, and more particularly to novel methods and apparatus for atraumatically diagnosing Peripheral Artery Disease (PAD) in subdermal tissue.

### BACKGROUND OF THE INVENTION

Peripheral Artery Disease (PAD) is a condition where atherosclerosis of the big arteries or other obstructive diseases affecting the small arteries compromise the ability of the circulatory system to deliver sufficient blood to peripheral regions of the body. The worldwide number of prevalent cases of PAD exceeds 200 million and the number of incident cases is over 10 million, with two thirds of prevalent cases of PAD being asymptomatic.

PAD is associated with significant morbidity and mortality. The compromised circulation may lead to chronic limb threatening ischemia (CLTI), a condition characterized by pain at rest, ulcers and wounds which do not heal. CLTI may lead to infection, tissue loss, and subsequent amputation. In addition, elevated prevalence of coronary artery disease, stroke, and hypertension are associated with PAD.

Vascular procedures and other treatments exist which can often improve the condition of PAD patients. However, the asymptomatic nature of the disease often means that treatment may not occur until after a patient develops CLTI and is at risk of amputation.

Many diagnostics for PAD have been proposed or are under development. None have advanced to the stage where they have significantly increased the detection of obstructive disease affecting big and small arteries, i.e., detecting the real tissue perfusion. Thus there is a need for non-invasive, inexpensive and quick diagnostics for PAD with a high degree of sensitivity and specificity in detecting tissue perfusion in order to identify PAD in its earliest stages, to evaluate the effectiveness of revascularization procedures, and to monitor the progression of the patient's disease.

Moreover, even when PAD is diagnosed, only time-consuming or expensive invasive diagnostics are currently available to identify and quantify the regions (e.g., angiosomes) with circulatory insufficiency. And even those diagnostics do not always highlight the locations most appropriate for treatment. For example, angiography produces a detailed map of the fluid in the arteries, but may not accurately reflect the flow passing through small collateral vessels connecting different angiosomes.

Thus, for invasive procedures, the physician must find the locations to treat by experienced guesswork, which may either prolong the procedure, if it takes time to find the best locations to treat, or leave some patients no better off, if those locations cannot be located. There exists the need for non-invasive, inexpensive diagnostics which can quickly and quantitatively highlight the circulatory health at locations a clinician examines in order to better plan and effect treatment of PAD.

Besides PAD patients, there are other medical needs which would be addressed by a non-invasive, inexpensive diagnostic which measures circulatory health at specific locations. For example, patients undergoing plastic or reconstructive surgery may have their vascular structures affected by the surgery, whether such structures are intentionally revised as part of the surgery or whether there is a risk that surgical intervention could compromise the health of vascular structures near the surgical field. There exists the need for a diagnostic to give quick, non-invasive feedback to the surgeon as to whether the circulation at locations in question are healthy after the surgical intervention.

Patients without PAD presenting systemic disease affecting connective tissue (e.g., scleroderma, rheumatoid arthritis, lupus erythematosus, etc.), or oedema, or cardiovascular conditions reducing cardiac output (e.g., heart failure, hypovolemia, septic shock, etc.) can present reduced tissue perfusion. Also in these cases there exists the need for a diagnostic to give quick, non-invasive feedback to the physician as to whether these conditions can affect the circulation at locations in question.

### SUMMARY OF THE INVENTION

One aspect of the invention is directed to a handheld probe that the operator may press against the patient. The probe has two parts which can move relative each other. The first part, which presses against the patient, has a blood circulation sensor near the tip contacting the patient which measures some aspect of the patient's circulation, e.g., the blood volume (i.e., the quantity of blood in the tissue region being sensed), the blood flow (i.e., the rate of flow of blood in the tissue region being sensed), or a pulsatile signal (i.e., the change in blood volume, or the change in blood flow, or the change in blood pressure, etc. in the tissue region being sensed). The operator holds a second part of the probe which is further away from the patient. The probe contains a means by which the second part transmits a force or pressure to the first part which is opposed by the patient, and a force or pressure sensor which provides a means of measuring the force exerted. The first part has a substructure with some defined shape whereby the force applies pressure to the patient. The probe further contains a means by which it can measure some aspect of the patient's circulation as a function of the applied pressure (e.g., an electronic controller connected to the circulation sensor and to the force or pressure sensor, with the electronic controller being configured to report the measured results to an observer and preferably also a data storage unit).

As used herein, the terms “force” and/or “pressure”, when used in the context of directing a force upon the anatomy using the apparatus of the present invention, refer to the force (or force per unit area, i.e., pressure) as applied by the operator to the probe of the present invention and, in turn, from one part of the probe to another part of the probe, and by the probe of the present invention to the anatomy of the patient. Since pressure is force per unit area, these terms can be easily converted from one to another and are used interchangeably in the application, as long as the effective area over which force is applied can be easily discerned. As used herein, the term “pulsatile signal” refers to a measured quantity having the same fundamental frequency as, and is in phase with, the heartbeat, and is generally correlated with the flow of blood pumped by the heart. By way of example but not limitation, the pulsatile signal can be a function of the volume, velocity, or flow rate of the blood in the body.

In one embodiment, the probe consists of a pair of telescoping cylindrical structures where the cylindrical structures are connected by a compression spring. The cylindrical structure contacting the patient has an atraumatic tip which contains a photoplethysmographic (PPG) sensor at the tip for sensing blood circulation. The operator holds the other cylindrical structure, which has a force or pressure sensor, and is able to apply pressure to the patient transmitted by the cylinders. The probe contains electronics which calculates the pulsatile signal from the PPG sensor while measuring the applied force via the force or pressure sensor. The pressure at which the pulsatile signal disappears or is significantly altered indicates the Tissue Perfusion Pressure (TPP) which is a significant marker of the circulatory health at the location where the probe contacts the patient. The absence of a pulsatile signal without any pressure being applied is a significant marker of extremely severe circulatory failure.

In another embodiment, the probe consists of two portions, with one portion carrying a blood circulation sensor (e.g., a PPG sensor) at its tip and with the other portion being configured to be held by the user so that the user can use the grasped portion to press the blood circulation sensor portion against a patient. A force or pressure sensor (e.g., a strain gauge) is provided on the probe to measure the force or pressure applied to the blood circulation sensor portion by the grasped portion.

In one form of the invention, there is provided an apparatus for performing a diagnostic measurement of blood circulation, wherein the apparatus comprises:
a probe with a tip that can be pressed against a patient;
said probe having a blood circulation sensor which measures the blood circulation in dermal and subdermal tissue near the tip when said probe is pressed against a patient;
said probe having a force or pressure sensor which measures the force or pressure applied by the probe to the patient;
wherein the force or pressure can be varied so an output of the blood circulation sensor may be assessed as a function of the force or pressure applied which is measured by the force or pressure sensor.

In one form of the invention, the force or pressure can be varied gradually so that a sufficiently precise TPP can be measured.

In another form of the invention, there is provided a method for performing a diagnostic measurement of blood circulation, wherein the method comprises:
providing an apparatus comprising:
   a probe with a tip that can be pressed against a patient;
      said probe having a blood circulation sensor which measures the blood circulation in dermal and subdermal tissue near the tip when said probe is pressed against a patient;
      said probe having a force or pressure sensor which measures the force or pressure applied by the probe to the patient;
      wherein the force or pressure can be varied so an output of the blood circulation sensor may be assessed as a function of the force or pressure applied which is measured by the force or pressure sensor;
pressing the tip of the probe against a patient;
using the blood circulation sensor to measure the blood circulation in dermal and subdermal tissue near the tip;
using the force or pressure sensor to measure the force or pressure applied by the probe to the patient; and
assessing the output of the blood circulation sensor as a function of the force or pressure applied which is measured by the force or pressure sensor.

In one form of the invention, the force or pressure is varied gradually so that a sufficiently precise TPP can be measured.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein:

FIG. 1 is a schematic view showing a pen-like probe formed in accordance with the present invention;

FIG. 2 is a schematic view of another pen-like probe formed in accordance with the present invention, wherein the probe comprises an electronic blood circulation sensor, an electronic force or pressure sensor, and a controller for receiving and processing the output of the blood circulation sensor and the force or pressure sensor, and also showing tissue pressure gradients under the probe tip for healthy and PAD patients which effectively stop the pulsatile flow;

FIG. 3 is a schematic view showing another pen-like probe formed in accordance with the present invention, with the pen-like probe comprising a laser diode source and photodiode receiver, and showing exemplary paths of photons emitted by the laser diode source which are incident on the photodiode receiver and which probe the blood flow in a tissue sample;

FIG. 4 is a schematic view showing another pen-like probe formed in accordance with the present invention, wherein the probe comprises a mechanical blood circulation sensor and a mechanical force or pressure sensor;

FIG. 5 are schematic views showing pulsatile signals acquired by the pen-like probe for patients with varying circulatory health;

FIG. 6 is a schematic view showing another pen-like probe formed in accordance with the present invention, wherein the probe comprises a force or pressure sensor in the form of a strain gauge;

FIG. 7 is a schematic view showing another pen-like probed formed in accordance with the present invention, wherein the probe comprises a gas-filled chamber and a force or pressure sensor in the form of a gas pressures sensor;

FIG. 8 is a picture showing a pen-like probe comprising a spring, a linear potentiometer, and blood circulation sensor module;

FIG. 9 is a picture showing a pen-like probe with a doppler blood flow sensor and pneumatic tube connected to a device that measures pulsatility and pressure;

FIG. 10 are schematic views showing tissue pressure gradients under the probe tip, with and without bone being disposed under the tip;

FIG. 11 is a schematic view showing a tissue sample and its blood flow being explored with an ultrasound probe;

FIG. 12 is a schematic view showing how a variety of different tips may be mounted to distal end of the probe so as to provide a desired tip feature and blood circulation sensor to the probe;

FIG. 13 is a schematic view showing how the probe may be mounted to a fixture; and

FIG. 14 shows measurements taken with the apparatus of FIG. 8;

FIG. 15 are schematic views of tissue pressure and perfusion measurements in (i) an ideal measurement (top section of figure), and (ii) an actual measurement (bottom section of figure); and

FIG. 16 are schematic views of how the actual measurements in the bottom section of FIG. 15 can be analyzed.

### DETAILED DESCRIPTION OF THE INVENTION

A variety of sensors have been used to assess the health of a patient's blood circulation. One of the most common is the finger pulse oximeter, which measures optical transmission at two wavelengths and its periodic variation with the patient's pulse, to measure the oxygen saturation of blood flowing to a patient's fingertip. Ultrasound is also used to measure blood flow, with Doppler ultrasound measuring blood velocity. X-ray imaging combined with contrast agents injected into a patient's arteries produces angiograms with highly resolved images of the fluid in the arteries.

Transcutaneous pO₂ sensors can sense oxygenation through the skin after heating the skin to allow oxygen to permeate the skin. Skin perfusion pressure (SPP) sensors measure the effect of pressure on blood circulation using one of three different sensing techniques for SPP measurement, i.e., radioisotope clearance, PPG or laser Doppler. Hyperspectral imagers in combination with image processing can image blood vessels and the degree of blood oxygen saturation. Laser speckle single pixel or image sensors are another means of measuring blood flow.

Another sensing methodology measures the ankle brachial index (ABI) by taking the ratio of systolic blood pressure at the ankle to that at the arm, which gives an assessment of blood circulation at the foot. Toe pressure and toe brachial index are considered more accurate, because this measurement gives an assessment of blood circulation in the extreme peripheral tissues.

While all of these sensors and methodologies provide useful information, they generally fall short in providing enough information to diagnose when a patient has PAD. While a measure using the ABI is correlated with PAD, it is not very sensitive or specific in predicting whether a patient has PAD, especially in patients with CLTI. ABI detects obstruction in the big arteries above-the-ankle, but is unable to detect small artery disease below-the-ankle. Toe pressure can be evaluated only in the first toe (i.e., the so-called “big toe”), but it cannot be applied in other sites of the foot or the body. In other cases, the various methodologies may measure a single quantity accurately, over a limited portion of the patient's tissue, but that may not be sufficient to diagnose PAD or to localize it to a specific blood vessel which can be targeted for surgical intervention to improve blood circulation.

For example, angiography provides detailed maps of the circulatory system that allow measurement of the physical size of blood vessels large enough to be imaged distinctly. However, such images may not predict the sufficiency of blood flow due to the inability to map small collateral vessels which feed various angiosomes. Several of the techniques only provide reliable measures of blood circulation near the skin, but subdermal tissues may experience different perfusion. The body's skin is subjected to the thermoregulatory function, and every measurement of its perfusion requires a preliminary time for the patient to adapt to the ambient temperature. Many of the techniques do not enable the measurement to be taken in volumes small enough or in arbitrary positions on the patient in order to localize circulatory problems to specific blood vessels or angiosomes.

Moreover, many of these techniques require the application of sensors and a pressure cuff on the skin of the patient (e.g., the SPP, PPG, ABI, and toe pressure techniques), leading to the need for resterilization of the device before using it on another patient.

Thus there exists a need for a device which can assess blood circulation within subdermal tissues that is not affected by, or is only partially affected by, thermoregulation, and which can probe a relatively small volume of tissue in an arbitrary position on the patient in order to diagnose PAD and localize circulatory problems to specific blood vessels or angiosomes. The device is a handheld probe (or fixture mounted probe) that can easily be covered with a sterile, disposable cover, avoiding the risk of infection transmission and the time for resterilization.

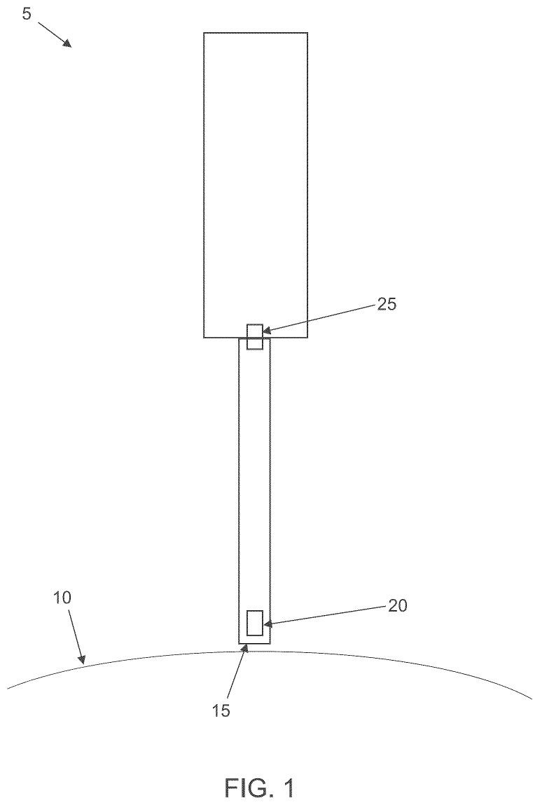

A device which overcomes these limitations is pictured in FIG. 1. A probe 5, preferably small enough to be handheld, is pressed against the patient's skin 10. The tip 15 of the probe contains a blood circulation sensor 20. The probe also contains a force or pressure sensor 25 to measure the force or pressure applied to the patient. The probe outputs a measure of blood circulation and the corresponding pressure applied to the patient. As will hereinafter be discussed, blood circulation sensor 20 can be electrical and/or mechanical and force or pressure sensor 25 can be mechanical and/or electrical.

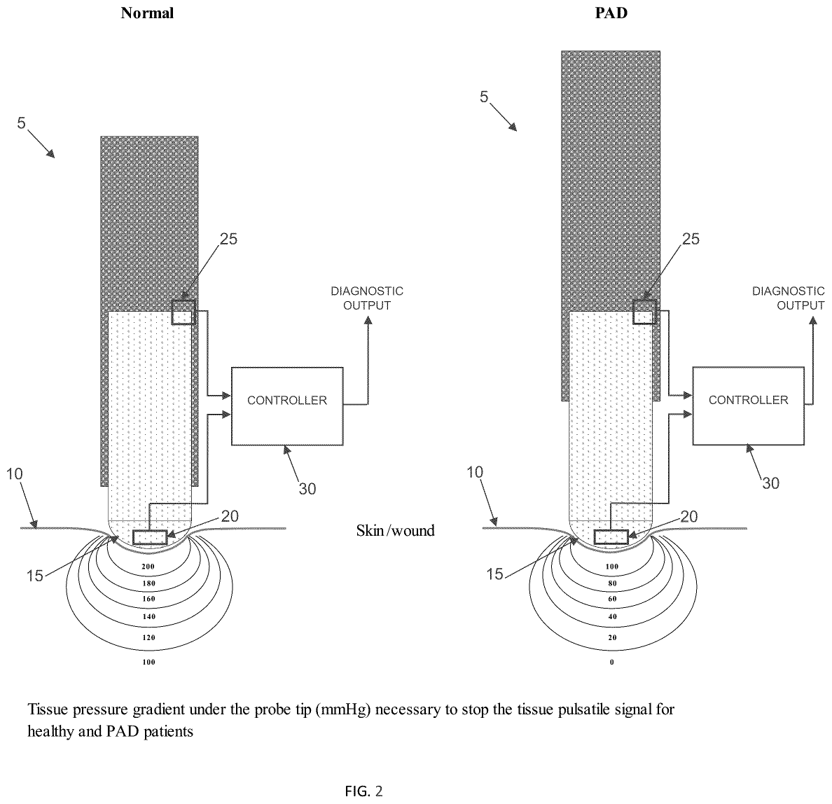

In one preferred form of the invention, and looking now at FIG. 2, blood circulation sensor 20 and force or pressure sensor 25 are both electrical, and their outputs are connected to a controller 30, which reports the measured results to an observer and preferably also a data storage unit (not shown).

As shown in FIG. 2, the pressure within the patient's tissue has a gradient where the pressure is highest close to the probe which falls off away from where the probe contacts the patient.

As the applied pressure increases, the blood is squeezed out of the vessels. Initially, the low pressure vessels (venous and capillary) are emptied of blood. With increasing pressure, the pulsatile flow of the arterial and arteriolar vessels is progressively overwhelmed. When the tissue pressure reaches the maximum pulsatile pressure of arteries and arterioles, the pulsatile signal disappears. Due to the pressure gradient, not every portion of the scanned tissue sample experiences the same pressure and hence there may not be a distinct pressure where the pulsatile signal disappears.

Nevertheless, a measure of how the blood circulation changes with applied pressure may be constructed that indicates the health of the patient's circulation. For instance, at high enough applied pressure, blood circulation measured by the blood circulation sensor 20 will fall below a defined threshold value. This threshold occurs at lower pressure for patients with PAD. This threshold pressure is termed the TPP. The TPP is greater for individuals with healthy circulation and less for individuals with PAD. In case of severe PAD, the pulsatile signal can be absent in the tissue circulation, and TPP is zero. In case of healthy subjects, in some areas of the body the pulsatile signal cannot be set to zero by any applied pressure, because blood continues to flow in bones and other non-compressible areas. The same phenomenon is noted with ABI, where the threshold of a healthy ABI is one where the ankle pressure is ≥0.9 times the arm pressure and lower indices are correlated with PAD. This effect of lower TPP for patients with PAD is illustrated in FIG. 2, where the TPP pressure is less for a patient with PAD, as are the associated pressure gradients, and zero for patients with severe PAD.

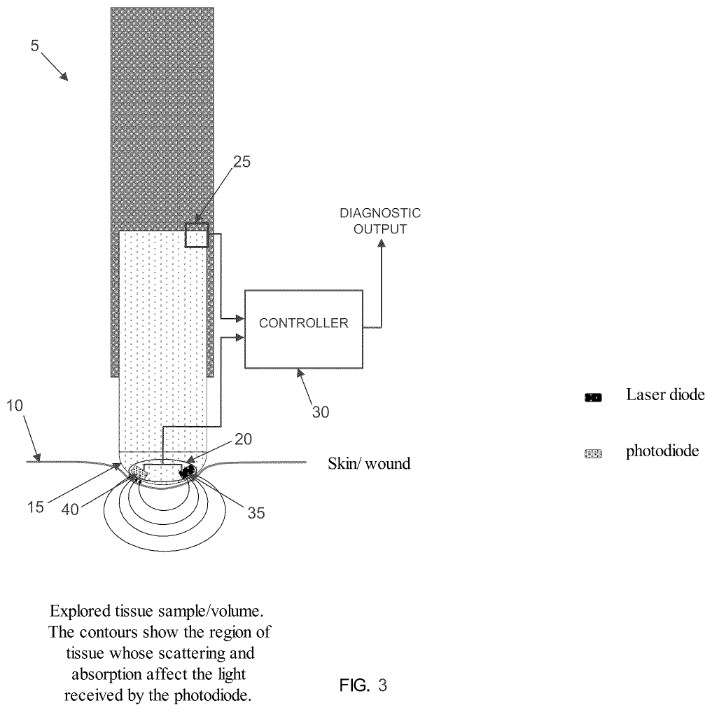

The observation that the pressure at which a measure of circulation falls below a threshold provides information about PAD is not new, nor is the concept of measuring said pressure. However, previous devices in the literature use a pressure cuff that surrounds a limb to apply pressure, and many are only capable of measuring that pressure at the skin, not in deeper tissue. The new probe of the present invention differs in that the pressure is applied locally. Thus it may provide more detailed information about the patient's blood circulation at a particular position on the body. As is pictured in FIG. 3, a blood circulation sensor 20 (e.g., a PPG sensor comprising a laser diode 35 and a photodiode 40) may be employed that senses blood circulation in tissue much deeper than the dermal layer.

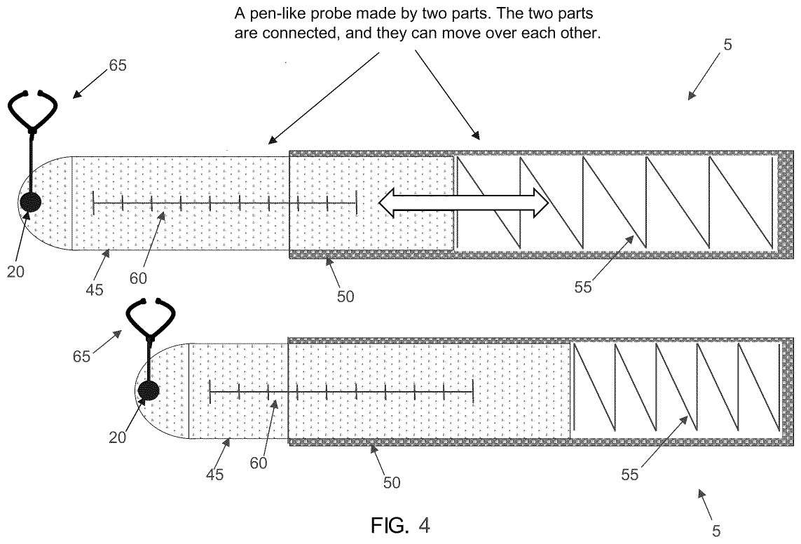

The variation of blood circulation with applied pressure measured by the probe may be observed differently depending on the particular sensors used and the configuration of the probe. In one embodiment, both the blood circulation sensor 20 and the force or pressure sensor 25 may provide output that is mechanically linked to the two sensors 20, 25 (e.g., the two sensors 20, 25 may be mechanical in nature). For example, and looking now at FIG. 4, the probe may be divided into two parts which are mechanically linked by a spring or other compressible mechanism, e.g., an inner tube 45 and an outer tube 50, with a spring 55 being used to bias the inner tube 45 distally. As pressure is applied, the inner tube 45 slides up into the outer tube. A visual scale 60 on the exterior of the inner tube 45 may be used to display a measure of the force/pressure applied. Furthermore, in this form of the invention, the blood circulation sensor 20 may have an acoustic output 65, shown schematically as a stethoscope, where the operator hears the pulsatile sounds coming from the scanned tissue sample during the examination. The operator can then sense the pressure at which the pulsatile signal disappears, just as is done with a sphygmomanometer to measure systolic blood pressure.

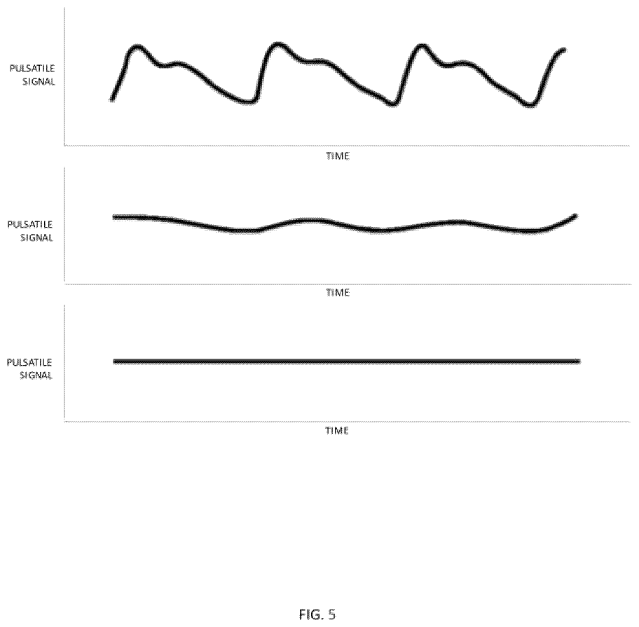

In other configurations, and more preferably, both the blood circulation sensor 20 and force or pressure sensor 25 may be electronic and their output may be mediated by electronic circuitry, e.g., a controller, and output as a visual or auditory signal. For instance, consider the sensor pictured in FIG. 3, which comprises an electronic blood circulation sensor 20 (i.e., a PPG sensor comprising a laser diode 35 and a photodiode 40) and an electronic force or pressure sensor 25 (e.g., a potentiometer for measuring linear displacements of one member relative to another member, or a strain gauge for measuring the force applied by one member to another member, etc.), with the outputs of blood circulation sensor 20 and force or pressure sensor 25 being fed to a controller 30, which is configured to provide an assessment of blood circulation as a function of the applied pressure or force. The laser diode 35 and photodiode 40 may comprise a PPG sensor which is similar to a reflective pulse oximeter. The photodiode signal may be digitized with an analog-to-digital converter (ADC). If sampled at a high enough rate, e.g., >5 Hz, the resulting time series will show a pulsatile signal similar to those shown in FIG. 5. In general, a signal from a circulation sensor will consist of a smaller variable or AC signal that modulates a larger positive or DC signal.

The time-sampled signal may be analyzed by a microprocessor or other computer to measure the circulation. A simple measure is the peak-to-valley modulation of the AC signal, either the absolute value or the fractional value as a ratio to the time-averaged or DC value of the signal. The force or pressure sensor 25 may also be digitized with an ADC. If the applied force/pressure is varied slowly enough, i.e., on a timescale measured in seconds, the measure of circulation may be assessed as a function of the applied force/pressure. The pressure at which the measure of circulation falls below an appropriate threshold then becomes the TPP for that location on the patient. That threshold may be a fixed threshold, either of the absolute or fractional value of a particular measure. It may also be a proportional threshold of an initial value. Consider a PPG signal, as shown in FIG. 5, where the top trace shows a baseline signal without pressure applied by the probe tip in the tissue sample, the middle trace shows less modulation when the applied pressure is reaching the tissue perfusion pressure, and the bottom trace shows the lack of a pulsatile signal regardless of applied pressure, as may occur in patients with very poor circulation. With no pressure applied, the peak-to-valley modulation is X % of the mean value. When pressure is applied, the peak-to-valley modulation decreases. A proportional threshold might be when the modulation drops to Y % of the initial modulation X %.

Other measures of the blood circulation may be used in order to define the TPP. The integral of the sensor signal over a fixed time period or over some number of heartbeats, or the integral of the sensor signal above a fixed or proportional threshold, may be used. In addition, a measure of the shape of the curve may be used. In FIG. 5, the top trace shows a healthy pulsatile signal showing a clear dicrotic notch with much high-frequency content. After applying pressure, the middle trace is much more sinusoidal without much high-frequency content. Patients with PAD may also exhibit pulsatile signals like the middle trace when no pressure is applied. In some cases of severe PAD, no pulsatile signal can be detected as is shown in the bottom trace. Various measures which quantify aspects of the shape of the time series are known to those skilled in the art, including those which measure the frequency content. These measures, either in an absolute or proportional sense, may be used in determining the TPP.

Since there will be pressure gradients within the tissue from application of pressure by the probe, and the circulation sensor measures over a volume of tissue, the measured blood circulation may not exhibit a very sharp cutoff with increasing pressure. Rather, it is likely to decrease more gradually. Thus some clinical data may be required to establish a measure of circulation and an appropriate threshold for a TPP that exhibits a high degree of sensitivity and specificity for diagnosing PAD or identifying blood vessels most affected by the disease.

The probe may be configured to produce an electronic output that signifies the sensor measurements or derived quantities. For instance, a speaker may output a tone whose pitch or volume is correlated with the output, or a display may show either a digital number or some other visual signal, e.g., color or intensity of a light-emitting diode (LED) or lamp reflecting the output. The outputs may also be sent electronically, either by wired or wireless connection, to a computing device which has a user interface (UI) that displays the measurements. The computing device may be a computer, tablet, or smartphone. When a derived quantity, such as the peak-to-valley modulation measurement, must be calculated from the data, that calculation may either be done by an embedded processor in the device or by a separate computing device.

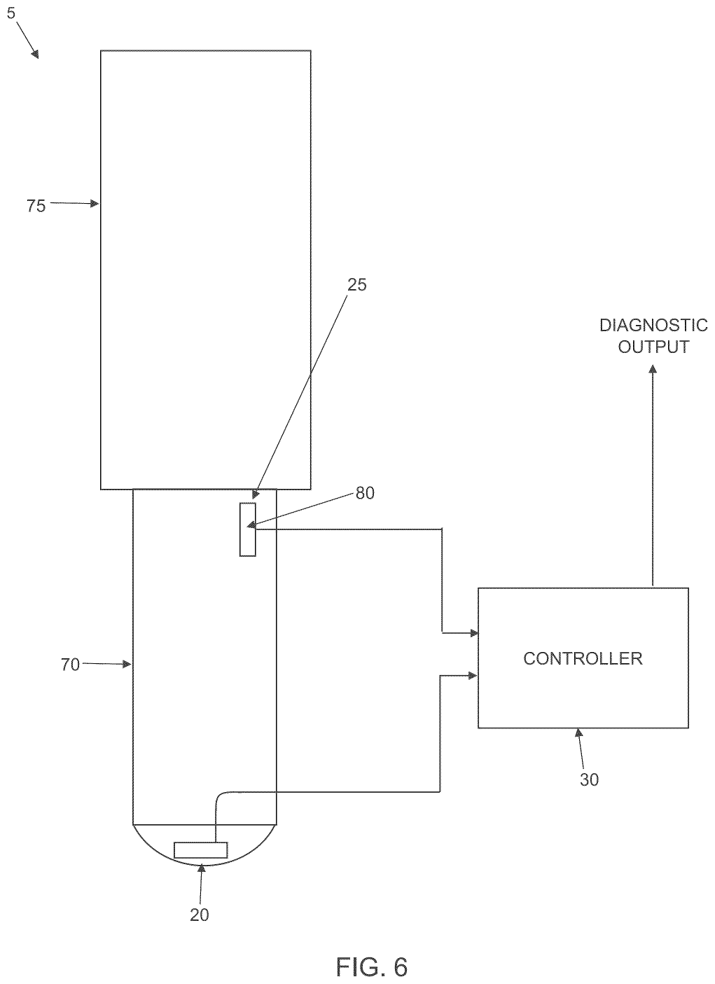

For embodiments where the force or pressure does not result in a mechanical indication, the probe may employ an electronic transducer that senses either the applied force or pressure. The output of that transducer (i.e., the output of force or pressure sensor 25) can then be reported on a computer or smaller device display. For instance, a strain gauge is a sensor whose output typically reflects the strain on a thin foil which can be related to the applied force. Similarly, force sensitive resistors produce an output related to the applied force. In both of these cases, the tip of the probe must be rigidly linked to the sensor. See, for example, FIG. 6, where a probe shaft 70 is securely mounted to a probe handle 75, and a strain gauge 80 measures the applied force.

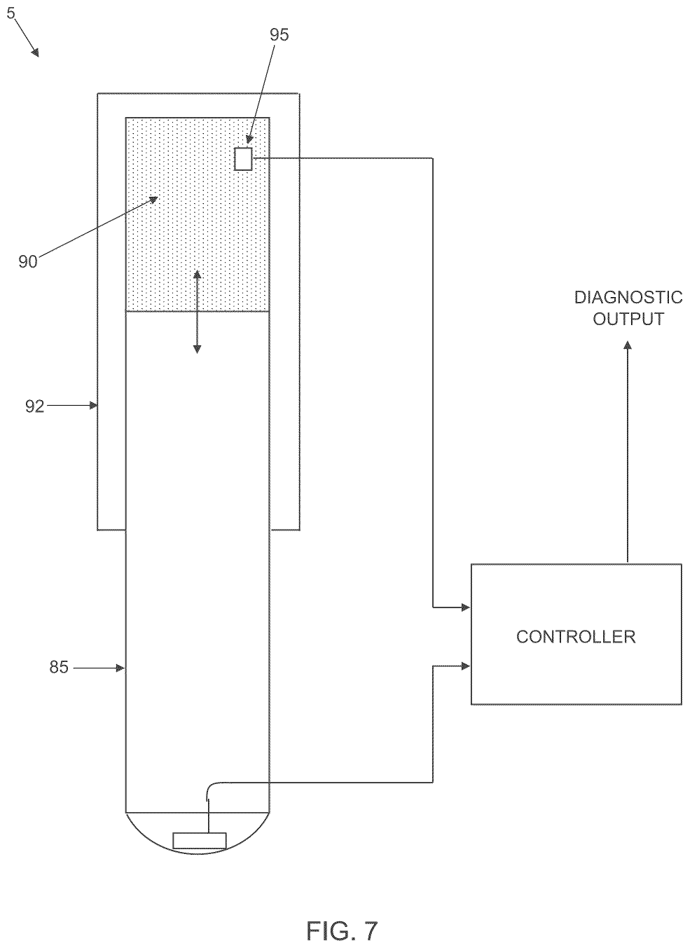

Another type of sensor is a pressure sensor which typically senses the deformation of a membrane by a pressure transmitted by a fluid, either liquid or gas, which is in contact with the membrane. This embodiment generally requires the tip of the probe push a piston in a cylinder whose contents come under pressure. See, for example, FIG. 7, where a probe piston 85 is slidably received in a gas-filled chamber 90 in a probe handle 92, and a gas pressure sensor 95 measures the pressure of the gas in gas-filled chamber 90, whereby to measure the force being applied to the patient by the probe.

Alternatively, and as seen in FIG. 4, force or pressure can also be sensed by measuring the displacement of a part of the probe which is connected to a second part of the probe by a compressible member such as a spring. A variety of means may be employed to measure the displacement.

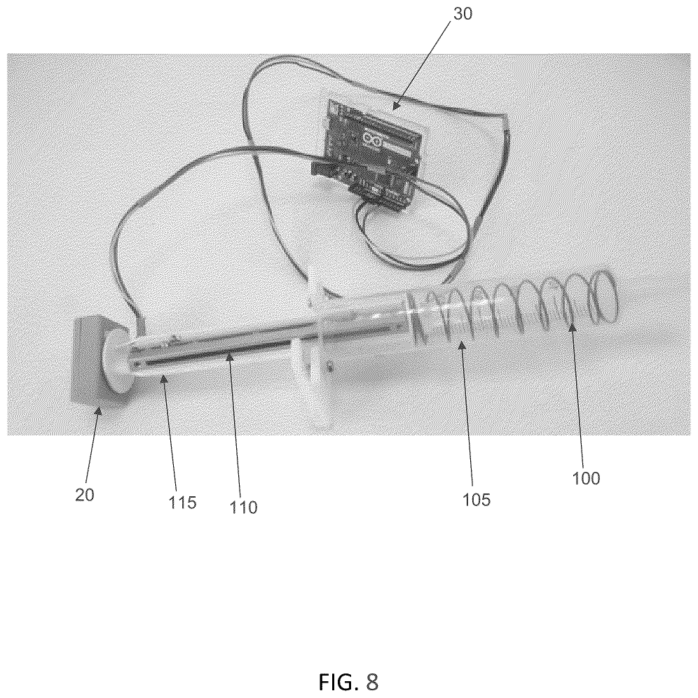

One embodiment senses the displacement of the sliding contact on a potentiometer by measuring the varying resistance to that contact. FIG. 8 shows an embodiment of a probe with a spring to set the force depending on the position of the plunger inside the syringe and a linear potentiometer to measure the position of the plunger and hence the force. The box on the opposite end of the plunger has laser diodes and photodiodes to detect perfusion under the pen. More particularly, FIG. 8 shows a pen-like probe with a spring 100 in the syringe barrel 105 to set the force and a linear potentiometer 110 on the plunger 115 to measure the position and hence the force. In addition, a blood circulation sensor 20 comprising a PPG sensor with laser diodes and photodiodes (not shown) is mounted to the end of the syringe plunger. The outputs of blood circulation sensor 20 and linear potentiometer 110 (which functions as the force or pressure sensor 25) are connected to controller 30, which provides an assessment of blood circulation as a function of the applied pressure or force.

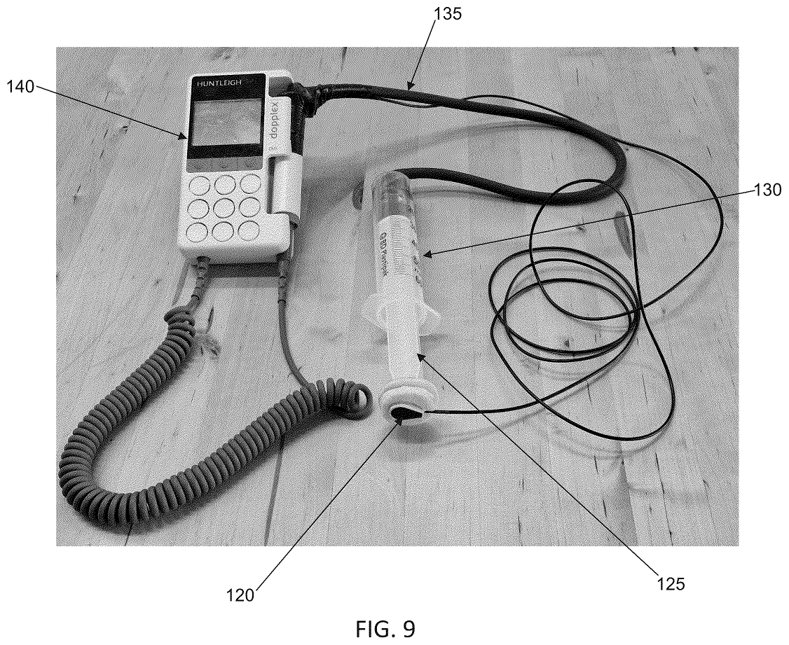

Another embodiment uses a syringe for a similar purpose. FIG. 9 shows a commercial doppler sensor with toe pressure cuff, the Huntleigh Dopplex DMX, adapted to measure TPP. The doppler probe on the end of the syringe plunger generates a pulsatile reading visible on the screen, while the pressure in the syringe is read by the mechanism used for the toe cuff. In one embodiment, the doppler probe or sensor 120 is mounted on the end of the syringe plunger 125 while the pressure in the syringe barrel 130 is communicated via a tube 135 to the toe pressure sensor 140. The device displays the measured pulsatile signal and pressure.

Some calculation may be required to equate the measured force or pressure to the pressure applied by the probe. This is a result of two factors. First, the applied pressure will depend on the area over which the probe contacts the patient and can apply a force to the skin. Second, it will depend on the properties of the materials used in the probe, especially at the tip. It is desirable that applying the probe to the patient causes no trauma, especially since patients suspected of having PAD may have wounds which do not heal quickly or at all due to circulatory insufficiency. Thus the materials chosen may be soft and compliant. While metals and hard plastics will undergo little deformation, the deformation of more compliant materials like rubber must be taken into account when calculating the pressure applied to the patient.

Additional factors may be required to calculate the pressure applied to the patient. For embodiments where force is measured, that force must be divided by the effective area of the tip of the probe. For embodiments where pressure is measured using a sliding piston-like member such as the probe piston 85 shown in FIG. 7, the measured pressure must be corrected for the ratio of the effective area of the tip of the probe to the area of the sliding piston-like member.

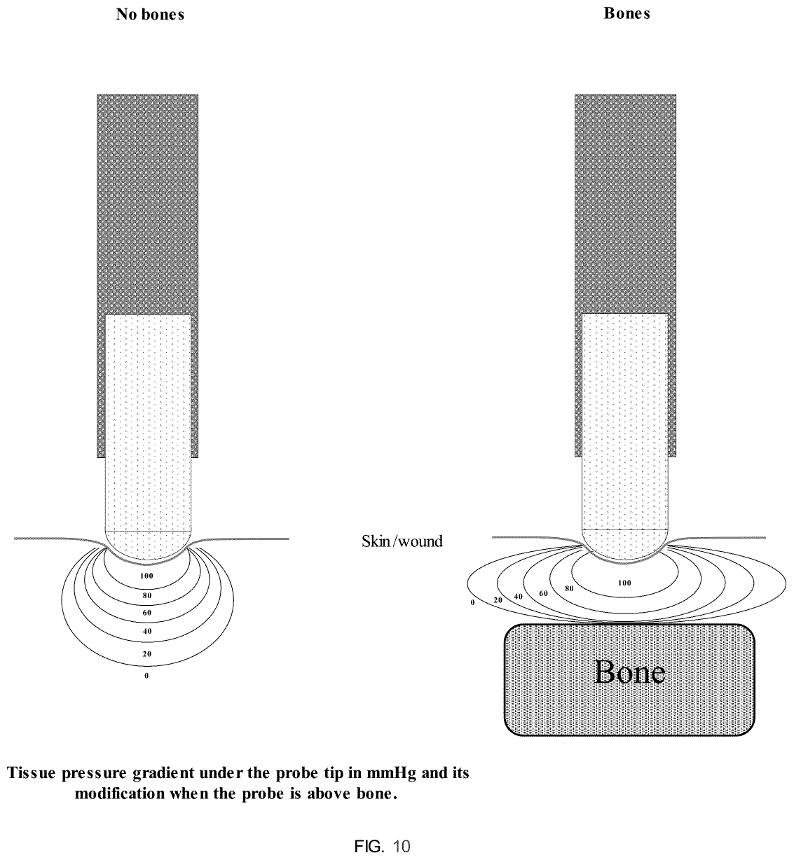

Additionally, the measurements made should account for the pressure gradients which will be produced in the patient's tissue. As shown in FIG. 10, the pressure gradients will vary depending on the type of tissue which the probe is pressed against. As is shown in FIG. 10, the pressure gradient may extend over a larger distance with soft tissue as compared to the gradient when a bone is close to the location where the probe is applied.

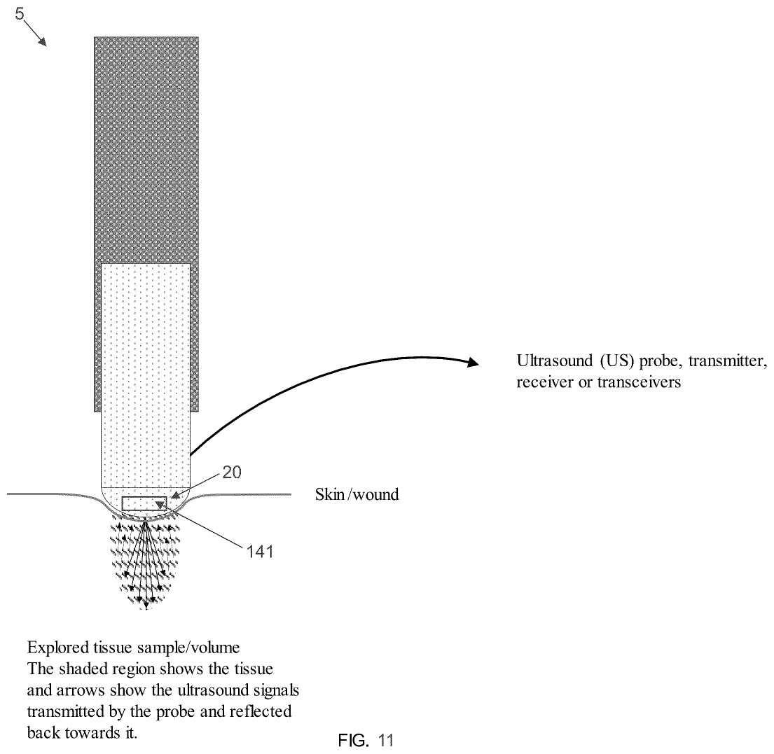

It is notable that the devices pictured in the Figures may employ a variety of blood circulation sensors 20. The blood circulation sensor 20 at the tip of the probe may be chosen from the range of sensors including ultrasound, laser Doppler, tcpO₂, SPP, PPG, hyperspectral imagers, and laser speckle. FIG. 11 shows a device with an ultrasound sensor at the tip (i.e., in this form of the invention, the blood circulation sensor 20 comprises an ultrasound sensor 141).

Depending on the location on the patient, it may be preferable to have a tip of a specific size and shape and made of a particular material. For some locations on the body, a tip with a flattened shape may be more suitable while for others, a rounded tip of a specified radius may be advantageous. For example, when probing around the hand or wrist or the bones of the foot, a probe with a smaller tip may afford greater precision in measuring the TPP. For areas where the underlying tissue is more uniform over several centimeters of lateral extent, a tip with a large flat region may be preferred.

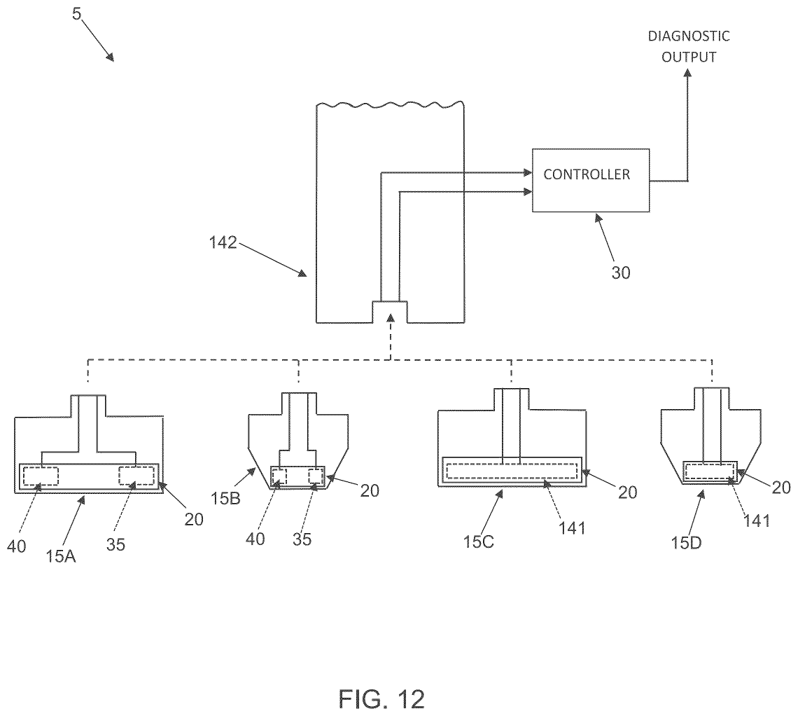

It is possible to engineer the probe so such tips with the appropriate shape, size, and detector configuration may be installed by the device operator just prior to measuring a particular location on a patient. For example, the PPG sensor shown in FIG. 3 may have a laser diode and photodiode mounted on a flat surface near the end of the probe. A removable tip may then attach to that surface which contains light guides which direct the laser diode output and photodiode input to and from regions on the surface of the tip which is pressed against the patient. Different tips may have different shapes to that surface, i.e., flat or rounded, and the distance between the light guides on the surface may be smaller or larger than the distance of the laser diode and photodiode on the fixed end of the probe to which the removable tip attaches. The operator may choose a different tip so that pressure applied by the probe is directed to a specific volume of tissue on the body appropriate to each location. See, for example, FIG. 12 which shows how four different tips 15A, 15B, 15C and 15D may be interchangeably mounted to the body 142 of probe 5 so as to provide a desired tip feature and a desired blood circulation sensor 20 to the probe. In this example, tips 15A and 15B use a PPG sensor (comprising a laser diode 35 and a photodiode 40) to provide the blood circulation sensor 20, but tips 15A and 15B have different surface profiles and/or different surface materials. And in this example, tips 15C and 15D use an ultrasound sensor 141 to provide the blood circulation sensor 20, but tips 15C and 15D have different surface profiles and/or different surface materials.

There is particular advantage to sensors which probe deeper than the skin layer, as the majority of circulation occurs below the skin and a device measuring circulatory health ideally probes the majority of circulation. In general, sensors which probe deeper than the skin layer are more complex than those which can only sense perfusion in the skin layer. Laser doppler flowmetry sensors typically have source and detector, realized with fiber optics, separated by 0.25 mm, which limits the depth of sensing to 0.5-1 mm with a measurement volume of approximately 1 mm³.

One means of performing subdermal sensing uses reflective PPG sensors with the light source(s) and detector(s) placed a large distance apart. PPG sensors have been described by Hielscher et al. in U.S. Pat. No. 11,439,312 with source-detector distances up to 25 mm. U.S. Pat. No. 11,439,312 is incorporated herein by reference.

Such PPG sensors typically have a near infrared (NIR) light source (typically an LED or laser diode) and a light sensing detector (typically a photodiode). NIR wavelengths ranging from 650 nm to 950 nm are typically used as they fall within the NIR window where light has its maximum depth of penetration in tissue. These wavelengths also fall within the range where hemoglobin in blood has significant absorption, and straddles the isosbestic point where the absorption spectra of oxygenated ([HbO₂]) and deoxygenated ([Hb]) hemoglobin cross. These wavelengths thus provide a range of spectral information which can be used to reconstruct the absorption coefficient, reduced scattering coefficient, and concentrations of [Hb] and [HbO₂].

Operation of the PPG sensor is as follows, referring to the embodiment shown in FIG. 12. Light from laser diode 35 enters tissue disposed in front of the probe tip and is absorbed and scattered. A portion of the light is reflected towards photodiode 40. The distance between the light source and detector is set by their mechanical mounting, which is preferably determined by a housing, such as the body of tip 15A or 15B, or by a printed circuit board (PCB) to which the components are soldered which is in turn attached to the body of the tip.

The distance between light source and detector determines the range of depth of tissue which is interrogated by light sensed by the detector. In a preferred embodiment of the invention, the distance between light source and detector falls within the range of 5 mm to 25 mm, as the resulting depth of tissue measured by the probe is advantageous for sensing physiologically significant volumes of tissue.

The invention described herein provides a hand-held probe which uniquely combines subdermal circulatory sensing with application of local force or pressure to provide specific information about the local circulation.

It is well-known that detected light in reflective PPG sensors follows a banana-shaped path where the most probable depth of a scattered photon path is roughly half the source-detector distance, but some detected light samples a depth that is more than half the source-detector distance or equal to the full source-detector distance. Thus, in accordance with the present invention, to probe tissue below the skin layers, a PPG detector is used which is constructed so that the source and detector are separated by 5 mm or more.

The light in such PPG sensors actually travels a distance much greater than the source-detector distance due to multiple scattering in the tissue, of order 5X the source-detector distance. As such, there is considerable attenuation of the light, and sensitive detection means must be employed to obtain a reasonable signal-to-noise ratio (SNR), especially since it is desirable to minimize the light power that shines into the body. Thus, in accordance with the present invention, in addition to using sensitive detectors and maximizing the optical coupling through the skin, sensitivity is preferably increased by using a lock-in detection scheme.

Lock-in detection involves modulating the source, sampling the detected signal at least at the Nyquist frequency of the modulation, and using the phase relationship between the samples and the modulation to estimate the signal while rejecting noise. A simple example uses on-off modulation and separately averages the signal when the source is on and off. The estimated signal is given by the difference which equals (background plus desired signal) minus (background). Other examples use sinusoidal modulation of the source with the detected signal multiplied by sine and cosine functions at the modulation frequency and these multiplied signals are summed to find the component of the detected signal (modulus and phase) at the modulation frequency which becomes the estimated signal. This multiplication can be done in analog fashion with mixers (multipliers) or digital multiplication of a sampled signal. The summation can similarly be done with analog integrators or by summing the multiplied sequences of the sine and cosine sequences (carrier frequency) and the detected signal.

Lock-in detection effectively narrows the bandwidth that contributes to the estimated signal to a small band of frequencies centered on the modulation frequency. The bandwidth is inversely related to the duration of the signal that contributes to each measurement estimate. Since the noise sources—e.g., electronic noise or background signals like room light—typically have a small fraction of their power at the modulation frequency, their noise contribution to the estimated signal is greatly reduced compared to a simple direct measurement of the signal at the detector. Such noise sources often have a component that arises from alternating current mains power (AC power) which is modulated at the AC power frequency or twice that frequency for rectified power. Therefore it is desirable that the lock-in detection modulation frequency exceeds twice the frequency of the AC power, or 100 or 120 Hz for most AC power worldwide.

In another preferred embodiment, the probe may comprise replaceable tips which include the circulatory sensor, whose parameters may be varied in order to better adapt to the target location. For example, the PPG sensor shown in FIG. 3 may have its end near the patient divided into two parts. The first part terminates with a printed circuit board (PCB) oriented normal to the length of the probe with connectors on the PCB. The second part is the tip which contacts the patient, which may have one or more sources and detectors which plug into the PCB connectors. A single probe may be configurable with multiple tips, each of which may differ in the number, size, and wavelength of its sources, the number, size, and performance of its detectors, or the spacing between sources and detectors. The appropriate tip may be selected depending on factors such how deep into the tissue it effectively probes. The effective depth will depend on factors such as (i) the source-to-detector distance, since the banana-shaped path goes deeper into tissue as the source-to-detector distance increases, (ii) the wavelength chosen, as a wavelength more readily absorbed will result in an ensemble of detected photons having shorter paths, on average, than wavelengths which are less readily absorbed, etc.

This invention was conceived as a means by which an operator, e.g., a physician, would manually place the probe against a patient and manually vary the pressure applied to the patient. However, the role of the operator may be replaced by a fixture which holds the tip against the patient and by an automated mechanism in the probe which applies a force or pressure which is transmitted to the patient. The fixture may be a rigid mechanical structure attached to the furniture the patient rests upon or it may be straps or tape attaching the probe to the patient or the furniture.

Many mechanisms can be constructed which would apply the force or pressure. Mechanisms which are considered linear actuators could be mounted to the probe and exert a force on the fixture, thereby causing an opposing force to be transmitted to the patient. A linear actuator consisting of a motor turning a screw held by a nut would exert a force against the nut, with the force increasing or decreasing depending on the direction the screw is turned. Current going through a coil of wire exerts a force on a magnetic material passing through the inside of the coil proportional to the current and resulting magnetic field. Other mechanisms like an inflatable bladder apply pressure when they are constrained by a housing and the pressure exerted on the housing varies with the inflation pressure. Additional mechanisms known to those skilled in the art could be automated to apply a variable force or pressure of the sort required to measure the TPP.

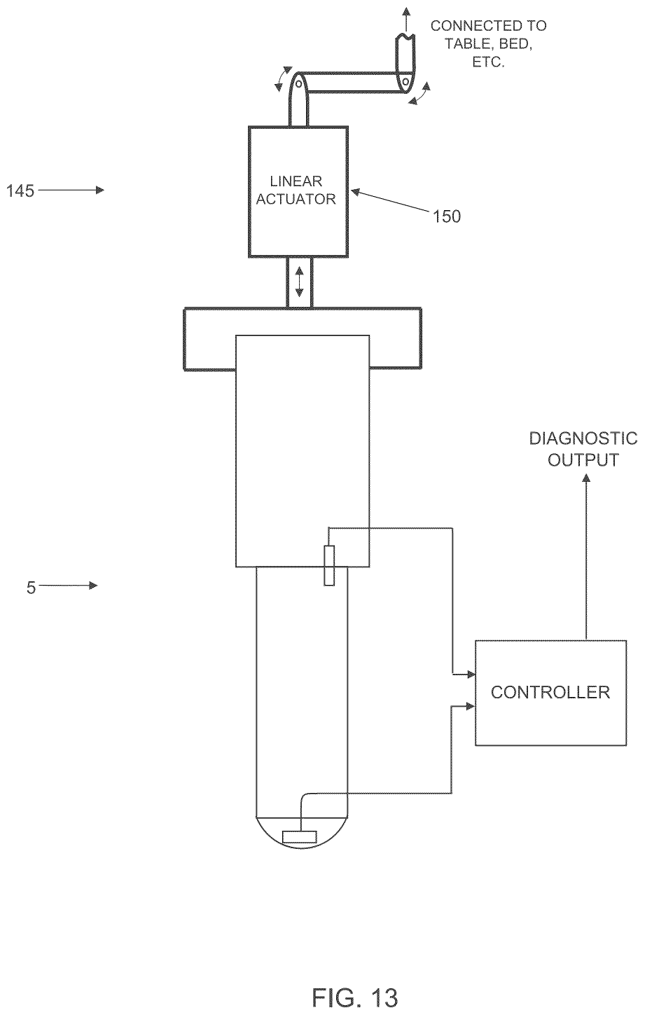

See, for example, FIG. 13 which shows probe 5 mounted to a fixture 145, wherein fixture 145 is mounted to a table, bed, etc. (not shown) and includes a linear actuator 150 for applying a force or pressure to the probe, which is then applied to the patient.

It will be appreciated that a fixtured, automated probe of the sort described need not be as large as one designed to be hand-held. A compact design could produce a probe that would not interfere with the actions of a surgeon who wanted to monitor the TPP at one or more locations during a procedure.

It should also be appreciated that probe 5 may be mounted to, or be formed as part of, a robotic arm, so that probe 5 can be manipulated robotically. In this case, blood circulation sensor 20 and force or pressure sensor 25 are preferably electrical, and their outputs are preferably transmitted along the length of the robotic arm to a controller 30 located proximal to the robotic arm.

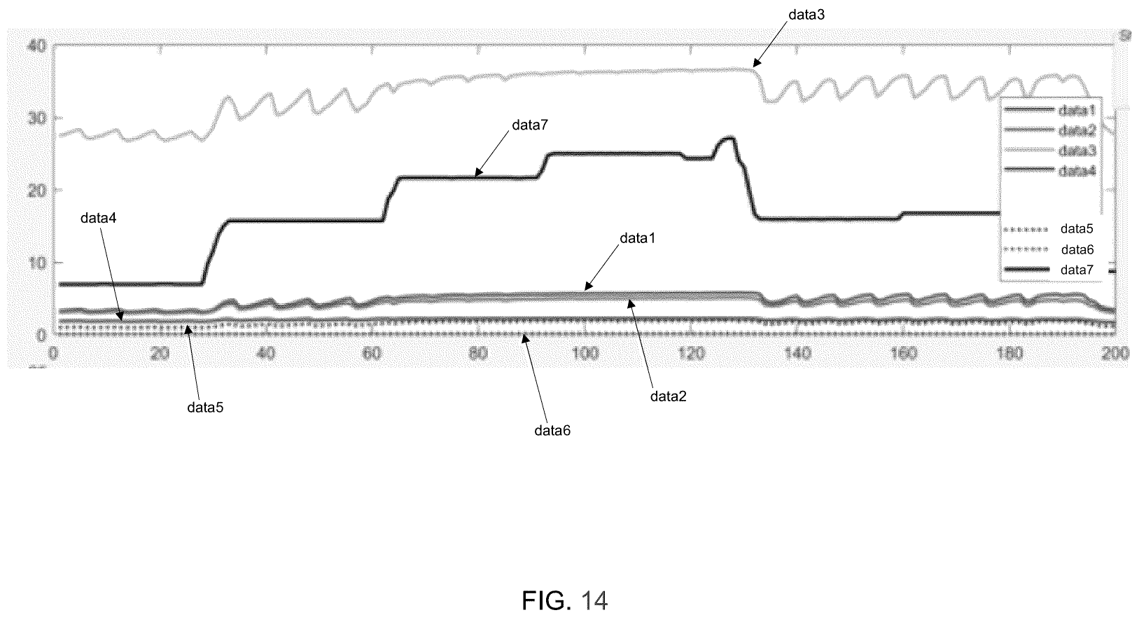

The results from two different prototype devices demonstrate the utility of this approach. Measurements taken with the device shown in FIG. 8 are displayed in FIG. 14. The colored traces labeled data1 thru data6 represent signals from a number of different laser diode-photodiode pairs on a sensor with 4 laser diodes and 2 photodiodes. The trace data7 measures the applied force on an arbitrary scale. The sampling rate is about 5 Hz. The probe was placed against a healthy patient with light force, at first, which was then increased 3 times. The amplitude of the pulsatile signal increased after full contact with the patient occurred from data points 30-65. The force was increased and then again after data point 90, and the pulsatile signal continued to decrease. When the force was reduced around data point 135, the pulsatile signal returned to its previous amplitude, thereby demonstrating a direct relationship between measured force and amplitude of the pulsatile signal.

The measurements displayed in FIG. 14 clearly show the ability of this invention to measure changes in circulation due to the applied force or pressure. Depending on the particular embodiment, measurements made with this invention may have a richer set of information than a single quantity such as the amplitude of the pulsatile signal or the TPP. For example, it is well known that PPG sensors with multiple source wavelengths can provide information about the oxygen saturation of blood. Pulse oximeters and more complicated devices to measure concentration of blood and its oxygen saturation rely on the fact that the near infrared (NIR) absorption spectra of oxygenated and deoxygenated hemoglobin cross at the isosbestic point near 808 nm. The device pictured in FIG. 8 has four laser diodes with wavelengths from 670 nm to 850 nm. The traces displayed in FIG. 14 show that the signal level changes as pressure is applied due to squeezing out some of the blood in the tissue. This reduces blood concentration and hence the absorption, thereby increasing the optical signal shown in FIG. 14. This effect is seen most clearly in the trace labeled data3. As disclosed in Hielscher et al. in U.S. Pat. No. 11,439,312, the device pictured in FIG. 8 can measure the concentrations of several chromophores such as oxygenated hemoglobin, deoxygenated hemoglobin, and water.

Other measures that can correlate with circulatory health may be constructed from the pulsatile signal, either by itself or in conjunction with other measurements. Recently, the pedal or plantar acceleration time (PAT), defined as the rise time of the velocity signal of blood flowing in a pedal or plantar artery observed with a duplex ultrasound sensor, is under study as a possible indicator of circulatory health. With sufficient resolution, and in accordance with the present invention, the pulsatile signals shown in FIG. 5 may be used to calculate (e.g., by means of a programmed controller provided with the probe 5) a measure that will correlate with the PAT. Most pulsatile signals will reflect either the velocity or volume of blood, and the acceleration is the derivative or second derivative of these quantities. In accordance with the present invention, such a measure may be combined with the force or pressure measurement (e.g., by means of a programmed controller provided with the probe 5) to indicate how this measure of circulatory health varies with applied force or pressure.

One example of such a provided feature would be the rise time of the pulsatile signal, i.e., the time to go from low to high, which will be referred to as the peak perfusion time (PPT). If signals like those seen in FIG. 5 are observed as the force or pressure increases, the PPT will increase with pressure. The pressure at which the PPT crosses a threshold may be a useful indicator. Such a determination may be made via a programmed controller provided with the probe 5. Another measure which may be provided with the present invention combines the amplitude of the pulsatile signal with the PPT. For example, the average slope of the rise of the pulsatile signal, which we will term the peak perfusion slope (PPS), would decrease with increasing pressure if signals like those seen in FIG. 5 are observed. Such a determination may be made via a programmed controller provided with the probe 5.

The device shown in FIG. 9 was used to measure the TPP on healthy patients as well as patients diagnosed with PAD. The TPP was assessed by gradually increasing the pressure exerted by the syringe and noting the pressure at which the pulsatile signal sensibly disappeared. The following tables show the TPP for healthy and PAD patients on the hand and foot.

Healthy Patient Hand TPP mmHg

| | Subject | Subject | Subject | Subject | Subject | | |
|---|---|---|---|---|---|---|---|
| site | 1 | 2 | 3 | 4 | 5 | mean | mean |
| 1° finger | 41 | 49 | 56 | 49 | 70 | 53 | 45 |
| 2° finger | 18 | 44 | 46 | 28 | 60 | 39 | |
| 3° finger | 24 | 44 | 50 | 36 | 68 | 44 | |
| 4° finger | 24 | 49 | 58 | 31 | 64 | 45 | |
| 5° finger | 19 | 49 | 45 | 30 | 64 | 41 | |
| Medial | 100 | 190 | 134 | 180 | 250 | 170 | 155 |
| palmar | | | | | | | |
| Lateral | 106 | 162 | 118 | 130 | 180 | 139 | |
| palmar | | | | | | | |

Healthy Patient Foot TPP mmHg

| site | Subject 1 | Subject 2 | Subject 3 | Subject 4 | mean |
|---|---|---|---|---|---|
| 1° toe | 72 | 78 | 55 | 58 | 66 |
| Medial plantar | 140 | 122 | 72 | 98 | 108 |
| Lateral plantar | 64 | 156 | 57 | 74 | 88 |
| Heel | 60 | 138 | 126 | 117 | 110 |

PAD Patient Foot TPP mmHg

| | Patient 1 | Patient 2 | | Patient 3 | |
|---|---|---|---|---|---|
| | PAD not | Revascularizable PAD | | Revascularizable PAD | |
| | revascular- | | Post- | | Post- |
| | izable | | revascular- | | revascular- |
| | baseline | baseline | ization | baseline | ization |
| 1° toe | 0 | 0 | 5 | 0 | 22 |
| Medial | 0 | 0 | 76 | 19 | 35 |
| plantar | | | | | |
| Lateral | 0 | 0 | 34 | 13 | 33 |
| plantar | | | | | |
| Heel | 0 | 0 | 36 | 0 | 100 |

It is clear from these measurements that the measured TPP as defined above and as measured at various locations with embodiments described herein shows a clear difference between healthy patients and those diagnosed with PAD. It also shows that patients who undergo a revascularization procedure show improved circulation as measured by the TPP.

The TPP is much higher for healthy patients than for PAD patients who have undergone a revascularization procedure. It is notable that for PAD patients, no pulsatile signal was measurable with the subject apparatus at some locations even with no pressure applied to the patient so the TPP at these locations was measured to be 0.

Other embodiments of this invention may be more sensitive and able to detect the pulsatile signal on these patients. This highlights the fact that measurements of TPP are likely to be device and location dependent. As is seen in FIGS. 2 and 10, we expect a gradient of pressures within the tissue and these gradients will differ depending on the tissue underneath the probe. Different sensor types and configurations will probe different portions of the tissue and hence the pressure gradient. The influence of these factors will affect the ability of a particular device to measure a TPP which can be correlated to other factors, like blood pressure, and which can be compared between different patients and location on the patient.

Although limited, the measured data show significant differences in the improvement from revascularization to different patients and significant variation at different locations. This is expected since multiple arteries supply the foot, and a revascularization procedure will not affect all arteries equally. This highlights the value of information TPP measurement with a device that can probe different locations can provide. TPP measurement can be used by a surgeon to identify locations or angiosomes with poor circulation which could benefit from intervention. It can also be used to quantify the improvement resulting from a surgical intervention. If this measurement can be made in real time during a surgical procedure, it offers the possibility of significantly improving surgical interventions. TPP measurement is non-invasive and is much less costly than the gold standard for assessing circulatory improvement during a procedure, i.e., x-ray angiography with contrast dyes.

The preceding discussion has focused on measurement of the TPP, defined as “pressure at which the pulsatile signal disappears or is significantly altered”, which is measured by varying the force or pressure applied by a probe to a specific location. Depending on how the pressure is applied and how the patient's body reacts, it may be difficult to hold the applied force or pressure sensibly constant to determine when the pulsatile signal disappears or is significantly altered.

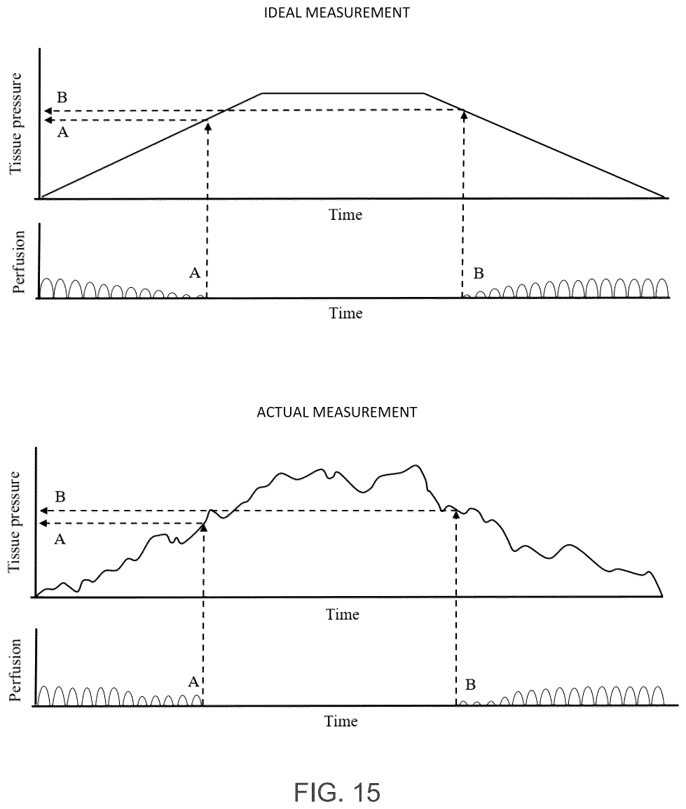

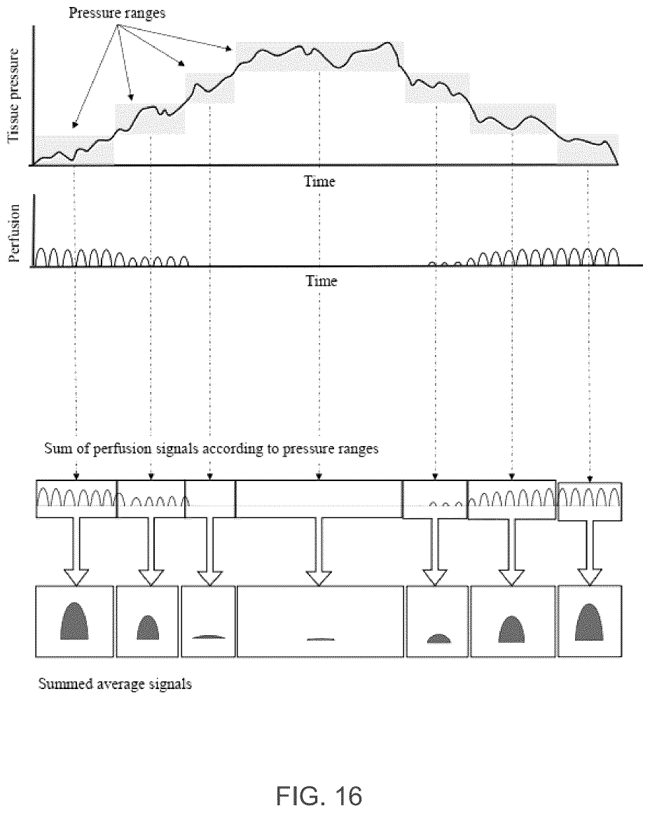

In accordance with the present invention, another strategy is to continually vary the applied pressure, either in a gradual manner or at a series of values set by the operator. The time sequence of circulatory measurements can then be “binned” into periods when the applied pressure falls within a given range or set of ranges. The pulsatile signal can then be assessed in each of these bins, e.g., by measuring the modulation of the pulsatile signal (peak-to-valley distance) about a mean change in the signal. FIG. 15 shows ideal data (top section), and more realistic exemplary data (bottom section) where the applied pressure does not vary linearly in an ideal fashion since the operator's control of the pressure fluctuates around the ideal curve. FIG. 16 shows the realistic exemplary data (top section of FIG. 16) binned into several discrete pressure ranges and the corresponding averaged pulsatile signals in each of those bins (bottom section of FIG. 16).

Although such binning with a gradually varying pressure may reduce the resolution of the estimated TPP, such a strategy may be more robust, overall, in determining the pressure range where significant variation of the pulsatile signal occurs. In fact, plotting a measure of the pulsatile signal strength (e.g., the amount of peak-to-valley modulation) versus these pressure bins may provide significant information about the circulatory health.

The “maneuver” (i.e., action) of compression and decompression of a body tissue sample with the handheld probe tip can be repeated several times while recording the tissue blood flow signals in the tissue sample. Subsequently, the recorded series of tissue blood flow signals will be divided and grouped in series of signals obtained at different specific pre-defined ranges of tissue pressure. The tissue pressure range at which the tissue blood flow signals are grouped can be the tissue pressure at the beginning of the blood flow signal or the mean tissue pressure during the blood flow signal, etc. Each group of blood flow signals or a processed measure of the blood flow may be appropriately summed in order to obtain an average tissue blood flow signal for a specific pressure range. If the blood flow signals are a pulsatile signal, each pulsation period may be synchronized before co-adding, or a measure such as the peak-to-valley modulation may be averaged across each pulsation period in a pressure bin. This process can be done automatically during the operator's maneuvers and can enable the apparatus to more accurately recognize the TPP than with a single maneuver.

Because the human body may react to the application of pressure in order to maintain its health, it is possible that the pressure at which the tissue blood flow signal disappears is different from the pressure at which the tissue blood flow reappears. The mechanical block of blood flow in the explored tissue sample could lead to engorgement of blood in the vessels surrounding the squeezed tissue sample, making them ready for reperfusion at a higher tissue pressure. This difference between compressive TPP and decompressive TPP is shown schematically in FIG. 15 (both top and bottom sections) and can have an important physiological meaning for PAD and CLTI evaluation.

There are other ways to operate the probe than applying a range of pressures until circulation effectively stops. In one form of the invention, the measure of perfusion, e.g., the amplitude of the pulsatile signal shown in FIGS. 14-16, can be measured at two or more pre-defined discrete pressures or pressure ranges. This approach can be facilitated by a probe which is configured to record measurements at a sequence of pre-defined discrete pressures or pressure ranges. As the probe cycles through two or more pressure levels, the probe can provide feedback to the operator so the operator can control the pressure to keep it close to the pre-defined value or appropriate range. One embodiment provides feedback in the form of a sound or tone that varies with the mismatch between the target pressure and actual pressure. The volume, frequency (pitch), or the speed of a sequence of beeps can all be varied to provide the feedback. Another embodiment provides feedback in the form of light. The brightness, color, and/or position of a light display about a center point can all provide feedback to guide the operator to control the applied pressure. Note that this feedback approach can also be applied to the measurement scenario where the pressure is gradually increased or decreased.

In the case of measurement at two discrete pressures, the relative change in the perfusion provides a measure of the circulatory health of the patient.

### Modifications

Although various exemplary embodiments of the invention have been disclosed, it should be apparent to those skilled in the art that various changes and modifications can be made which will achieve some of the advantages of the invention without departing from the true scope of the invention.

What is claimed is:

1. An apparatus for performing a diagnostic measurement of blood circulation, wherein the apparatus comprises:
a probe with a tip that can be pressed against a patient;
said probe having a blood circulation sensor which measures the blood circulation in dermal and subdermal tissue near the tip when said probe is pressed against a patient;
said probe having a force or pressure sensor which measures the force or pressure applied by the probe to the patient;
wherein the force or pressure can be varied so an output of the blood circulation sensor may be assessed as a function of the force or pressure applied which is measured by the force or pressure sensor.

2. The apparatus according to claim 1 wherein the force or pressure can be varied gradually so that a sufficiently precise Tissue Perfusion Pressure (TPP) can be measured.

3. The apparatus according to claim 1 wherein the blood circulation sensor measures at least one from the group consisting of blood volume, blood flow and a pulsatile signal.

4. The apparatus according to claim 1 wherein the blood circulation sensor which measures the blood circulation comprises a reflective photoplethysmography sensor comprising a light source and a light detector.

5. The apparatus according to claim 4 wherein the light source and the light detector of the blood circulation sensor are at least 5 mm distant as measured on the skin when pressed against a patient.

6. The apparatus according to claim 4 wherein the blood circulation sensor comprises at least two light sources.

7. The apparatus according to claim 4 wherein the blood circulation sensor comprises at least two light detectors.

8. The apparatus according to claim 4 wherein the blood circulation sensor comprises at least one light source, at least one light detector, and at least one source-to-detector distance, wherein the at least one light source, the at least one light detector, and the at least one source-to-detector distance are selected to optimize the average tissue depth targeted by the blood circulation sensor.

9. The apparatus according to claim 1 wherein the blood circulation sensor has a sample rate greater than 5 Hz.

10. The apparatus according to claim 4 wherein the apparatus employs a lock-in detection scheme.

11. The apparatus according to claim 10 wherein the lock-in detection modulation frequency is greater than 120 Hz.

12. The apparatus according to claim 5 wherein the apparatus employs a lock-in detection scheme.

13. The apparatus according to claim 1 wherein the probe comprises a first part and a second part, wherein the first part is movable relative to the second part, wherein the blood circulation sensor is carried by the first part, and further wherein the force or pressure sensor is configured to measure movement of the first part and the second part relative to one another.

14. The apparatus according to claim 13 wherein the force or pressure sensor comprises an element selected from the group consisting of a potentiometer, a strain gauge and a pressure sensor.

15. The apparatus according to claim 1 wherein the tip comprises a first tip having a first shape comprising a first material with a first blood circulation sensor, wherein the first tip is removable and may be replaced by a second tip which differs from the first tip, the difference comprising a feature selected from the group consisting of shape, material, and blood circulation sensor.

16. The apparatus according to claim 1 wherein the probe is held by a fixture so the tip contacts a patient and wherein the probe further comprises a means for applying a force or pressure to the patient.

17. The apparatus according to claim 1 further comprising a processor configured to electronically sample the blood circulation sensor and the force or pressure sensor during the duration of a measurement.

18. The apparatus according to claim 17 further comprising a processor configured to bin the blood circulation samples into periods defined by ranges of the force or pressure sensor.

19. The apparatus according to claim 18 wherein the processor is configured to calculate a measure of blood circulation in each of the periods defined by ranges of the force or pressure sensor.

20. The apparatus according to claim 17 wherein the processor is configured to calculate the deviation of the measured force or pressure from a target force or pressure, and further wherein the apparatus provides a feedback output to the operator that changes in response to that deviation.

21. The apparatus according to claim 20 wherein the processor is configured with at least two target force or pressure values, wherein the processor is further configured with at least two time periods corresponding to the target force or pressure values, and wherein the processor bins the blood circulation measurements from each of the at least two time periods.

22. A method for performing a diagnostic measurement of blood circulation, wherein the method comprises:
providing an apparatus comprising:
a probe with a tip that can be pressed against a patient;
said probe having a blood circulation sensor which measures the blood circulation in dermal and subdermal tissue near the tip when said probe is pressed against a patient;
said probe having a force or pressure sensor which measures the force or pressure applied by the probe to the patient;
wherein the force or pressure can be varied so an output of the blood circulation sensor may be assessed as a function of the force or pressure applied which is measured by the force or pressure sensor;
pressing the tip of the probe against a patient;
using the blood circulation sensor to measure the blood circulation in dermal and subdermal tissue near the tip;
using the force or pressure sensor to measure the force or pressure applied by the probe to the patient; and
assessing the output of the blood circulation sensor as a function of the force or pressure applied which is measured by the force or pressure sensor.

23. The method according to claim 22 wherein the force or pressure is varied gradually so that a sufficiently precise Tissue Perfusion Pressure (TPP) can be measured.

24. The method according to claim 22 wherein the blood circulation sensor measures at least one from the group consisting of blood volume, blood flow and a pulsatile signal.

25. The method according to claim 22 wherein the blood circulation sensor which measures the blood circulation comprises a photoplethysmography sensor comprising a light source and a light detector.

26. The method according to claim 25 wherein the light source and the light detector of the blood circulation sensor are at least 5 mm distant as measured on the skin when pressed against a patient.

27. The method according to claim 25 wherein the blood circulation sensor comprises at least two light sources.

28. The method according to claim 25 wherein the blood circulation sensor comprises at least two light detectors.

29. The method according to claim 25 wherein the blood circulation sensor comprises at least one light source, at least one light detector, and at least one source-to-detector distance, wherein the at least one light source, the at least one light detector, and the at least one source-to-detector distance are selected to optimize the average tissue depth targeted by the blood circulation sensor.

30. The method according to claim 25 wherein the blood circulation sensor has a sample rate greater than 5 Hz.

31. The method according to claim 25 wherein the apparatus employs a lock-in detection scheme.

32. The method according to claim 31 wherein the lock-in detection modulation frequency is greater than 120 Hz.

33. The method according to claim 26 wherein the apparatus employs a lock-in detection scheme.

34. The method according to claim 22 wherein the probe comprises a first part and a second part, wherein the first part is movable relative to the second part, and further wherein the blood circulation sensor is carried by the first part, and further wherein the force or pressure sensor is configured to measure movement of the first part and the second part relative to one another.

35. The method according to claim 34 wherein the force or pressure sensor comprises an element selected from the group consisting of a potentiometer, a strain gauge and a pressure sensor.

36. The method according to claim 22 wherein the tip comprises a first tip having a first shape comprising a first material with a first blood circulation sensor, wherein the first tip is removable and may be replaced by a second tip which differs from the first tip, the difference comprising a feature selected from the group consisting of shape, material, and blood circulation sensor.

37. The method according to claim 22 wherein the probe is held by a fixture so the tip contacts a patient and wherein the probe further comprises a means for applying a force or pressure to the patient.

38. The method according to claim 22 further comprising a processor configured to electronically sample the blood circulation sensor and the force or pressure sensor during the duration of a measurement.

39. The method according to claim 38 further comprising a processor configured to measure a quantity selected from the group consisting of the Peak Perfusion Time (PPT) and the Peak Perfusion Slope (PPS).

40. The method according to claim 38 further comprising a processor configured to bin the blood circulation samples into periods defined by ranges of the force or pressure sensor.

41. The method according to claim 40 wherein the processor is configured to calculate a measure of blood circulation in each of the periods defined by ranges of the force or pressure sensor.

42. The method according to claim 40 wherein the processor is configured to calculate the deviation of the measured force or pressure from a target force or pressure, and further wherein the apparatus provides a feedback output to the operator that changes in response to that deviation.

43. The method according to claim 42 wherein the processor is configured with at least two target force or pressure values, wherein the processor is further configured with at least two time periods corresponding to the target force or pressure values, and wherein the processor bins the blood circulation measurements from each of the at least two time periods.

44. The method according to claim 22 wherein the output of the blood circulation sensor is analyzed to determine the variation with force or pressure of at least one from the group consisting of blood oxygen saturation, total hemoglobin concentration, oxygenated hemoglobin concentration, deoxygenated hemoglobin concentration, and water concentration.
